(19) United States
(12) Patent Application Publication
(10) Pub. No.: US 2007/0161978 A1
(43) Pub. Date: Jul. 12, 2007

(54) Multifunctional medical device assembly

(76) Inventors: Adam S. Fedenia, Libertyville, IL (US); Peter L. Visconti, Gurnee, IL (US)

Correspondence Address: Kim Luna, 1430 Waukegan Road, McGaw Park, IL, 60085-6787, US

(21) Appl. No.: 11/594,976

(22) Filed: Nov. 9, 2006

Related U.S. Application Data
(60) Provisional application No. 60/735,165, filed on Nov. 10, 2005.

(51) Int. Cl. A61B 18/04

(52) U.S. Cl. 606/34

(57) ABSTRACT

Embodiments of the invention provide an integrated, disposable medical device assembly including a handpiece having suction, irrigation, and electrosurgical control structure, irrigation tubing configured for connection to an irrigation fluid source at one end and connected to the handpiece at another end, and suction tubing configured for connection to a source of suction at one end and connected to the handpiece at another end. The assembly may include a fluid pump and electrical wiring leading from the handpiece and configured for connection a source of electrosurgical energy.

### CROSS REFERENCE TO RELATED APPLICATIONS

This application claims the benefit of U.S. Provisional Application No. 60/735,165, filed Nov. 10, 2005, under 35 U.S.C. § 119(e). The entire disclosure of that provisional application is incorporated by reference herein.

### FIELD OF THE INVENTION

The invention relates generally to medical devices, such as, for example, medical devices that provide multiple functions during a medical procedure. More particularly, embodiments of the invention relate to a disposable assembly for a hand-held medical device providing suction, irrigation, cutting and/or coagulation capabilities.

### BACKGROUND OF THE INVENTION

A wide variety of hand-held medical devices are known in the medical field. Certain invasive surgical procedures utilize electrical-sourced cutting, ablating, coagulation, and/or cauterizing instruments. Within the particular treatment location, such as, for example, within an internal cavity of the patient's body undergoing treatment, such procedures can generate steam, vapors, and smoke from heated or burnt tissue. When this occurs, visualization of the surgical site can become obscured, leading to potential dangerous conditions for the patient.

During the course of performing invasive medical procedures, such as laparoscopic surgery, a variety of instruments and equipment are utilized. Certain procedures require the use of lavage devices, which cleanse the surgical site of debris, smoke, and fluids that obscure the practitioner's visibility of the procedure and site, or are otherwise undesirable. One example of such a device is commonly referred to as a suction-irrigation assembly.

Suction-irrigation assemblies typically include a handpiece, tubing, a fluid source, suction source, and a pump mechanism. These components are often purchased or provided individually or separate for user self-assembly. The mechanical and electrical compatibility of the various components with one another, as well as the reusability or disposability of the components, is a constant consideration that must be accounted for during set-up for the given procedure.

The required assembly and connection between individually manufactured components in prior systems creates an undesired burden on the users and staff. The medical device operators (and their staff) must ensure correct and complete set-up and operative success of the components relative to one another in order for the procedure to be successful. Furthermore, the users and staff must ensure that all of the separated components are in inventory and available, in addition to being mechanically and electrically compatible. This is especially important where the handpiece and remaining assembly includes not only the suction-irrigation functional capabilities, but also the electrosurgical capabilities, such as cauterization, cutting and coagulation functions as well.

Taking into account the problems noted above, there exists a need in the medical and surgical field for suction-irrigation assemblies that simplify preparation, presentation, use and disposal, for a given procedure.

### SUMMARY OF THE INVENTION

One embodiment of the invention is directed to an integrated, disposable medical device assembly including a handpiece having suction, irrigation, and electrosurgical control structure and irrigation tubing configured for connection to an irrigation fluid source at one end and connected to the handpiece at another end. The assembly further includes suction tubing configured for connection to a source of suction at one end and connected to the handpiece at another end and a disposable fluid pump located along a path of the irrigation tubing. A power pack is electrically coupled to the disposable fluid pump to provide power to the disposable fluid pump and electrical wiring leads from the handpiece and is configured for connection to a source of electrosurgical energy.

Another embodiment of the invention is directed to An integrated, disposable medical device assembly, including a handpiece having suction, irrigation, and electrosurgical control structure and irrigation tubing configured for connection to an irrigation fluid source at one end and connected to the handpiece at another end. The assembly further includes suction tubing configured for connection to a source of suction at one end and connected to the handpiece at another end and a disposable fluid pump cassette located along a path of the irrigation tubing. Electrical wiring leads from the handpiece and is configured for connection a source of electrosurgical energy.

Another embodiment is directed to a method of assembling and using a medical device including removing from packaging, an integrated, disposable medical device assembly. The medical device assembly includes a handpiece having suction, irrigation, and electrosurgical control structure and irrigation tubing configured for connection to an irrigation fluid source at one end and connected to the handpiece at another end. The assembly further includes suction tubing configured for connection to a source of suction at one end and connected to the handpiece at another end and a disposable fluid pump located along a path of the irrigation tubing. The assembly also includes a power pack electrically coupled to the disposable fluid pump to provide power to the disposable fluid pump and electrical wiring leading from the handpiece and configured for connection a source of electrosurgical energy. The method includes connecting the irrigation tubing to an irrigation fluid source, connecting the suction tubing to a source of suction, connecting the wiring to an electrosurgical generator, using the assembly to provide irrigation, suction, and electrosurgical treatment in a medical procedure, and disposing of the medical device assembly.

Another embodiment is directed to a method of assembling and using a medical device including removing from packaging, an integrated, disposable medical device assembly. The medical device assembly includes a handpiece having suction, irrigation, and electrosurgical control structure and irrigation tubing configured for connection to an irrigation fluid source at one end and connected to the handpiece at another end. The assembly further includes suction tubing configured for connection to a source of suction at one end and connected to the handpiece at another end and a disposable fluid pump cassette located along a path of the irrigation tubing. Electrical wiring leads from the handpiece and is configured for connection a source of electrosurgical energy. The method further includes connecting the irrigation tubing to an irrigation fluid source, connecting the suction tubing to a source of suction, connecting the wiring to an electrosurgical generator, connecting the cassette to a reusable pump system powered by an AC power source, using the assembly to provide irrigation, suction, and electrosurgical treatment in a medical procedure, and disposing of the medical device assembly.

Another embodiment of the invention includes a method of packaging and shipping a disposable medical device. The method includes packaging, an integrated, disposable medical device assembly. The medical device assembly includes a handpiece having suction, irrigation, and electrosurgical control structure and irrigation tubing configured for connection to an irrigation fluid source at one end and connected to the handpiece at another end. The assembly further includes suction tubing configured for connection to a source of suction at one end and connected to the handpiece at another end and a disposable fluid pump located along a path of the irrigation tubing. The assembly also includes a power pack electrically coupled to the disposable fluid pump to provide power to the disposable fluid pump and electrical wiring leading from the handpiece and configured for connection a source of electrosurgical energy. The method includes enclosing the assembly within packaging and shipping the enclosed assembly to a supplier or user.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a schematic diagram of an integrated, disposable suction-irrigation assembly with a battery powered pump, according to one embodiment of the present disclosure.

FIG. 2 is a schematic diagram of an integrated, disposable suction-irrigation assembly with a cassette compatible for an AC-powered pump, according to one embodiment of the present disclosure.

FIG. 3 depicts one example of a disposable assembly representing an embodiment corresponding to the schematic of FIG. 1, according to one embodiment of the present disclosure.

FIG. 4A depicts one example of a disposable assembly representing an embodiment corresponding to the schematic of FIG. 2, according to one embodiment of the present disclosure.

FIG. 4B depicts an alternative fluid supply for use in the embodiment of FIG. 4A, according to one embodiment of the present disclosure.

FIG. 5 is a disassembled perspective view of an exemplary medical device handpiece and an instrument for use in an assembly, according to an embodiment of the present disclosure.

FIG. 6 is a schematic diagram depicting an exemplary battery powered pump for use in an irrigation-suction assembly, according to an embodiment of the present disclosure.

FIG. 7A is a perspective view of an exemplary motor for use in the battery powered pump of FIG. 6, according to an embodiment of the present disclosure.

FIG. 7B is a cross-sectional view of an exemplary motor for use in the battery powered pump of FIG. 6, according to an embodiment of the present disclosure.

FIG. 8 depicts an AC-powered pump and a cassette compatible therewith, according to one embodiment of the present disclosure.

FIGS. 9A-9C depict connection of a cassette providing an irrigation flowpath to an AC-powered pump, according to one embodiment of the present disclosure.

### DESCRIPTION OF THE EMBODIMENTS

Reference will now be made in detail to the present exemplary embodiments of the invention illustrated in the accompanying drawings. Wherever possible, the same reference numbers will be used throughout the drawings to refer to the same or like parts. For purposes of this disclosure, “distal” refers to the end further from the device operator during use, and “proximal” refers to the end closer to the device operator during use.

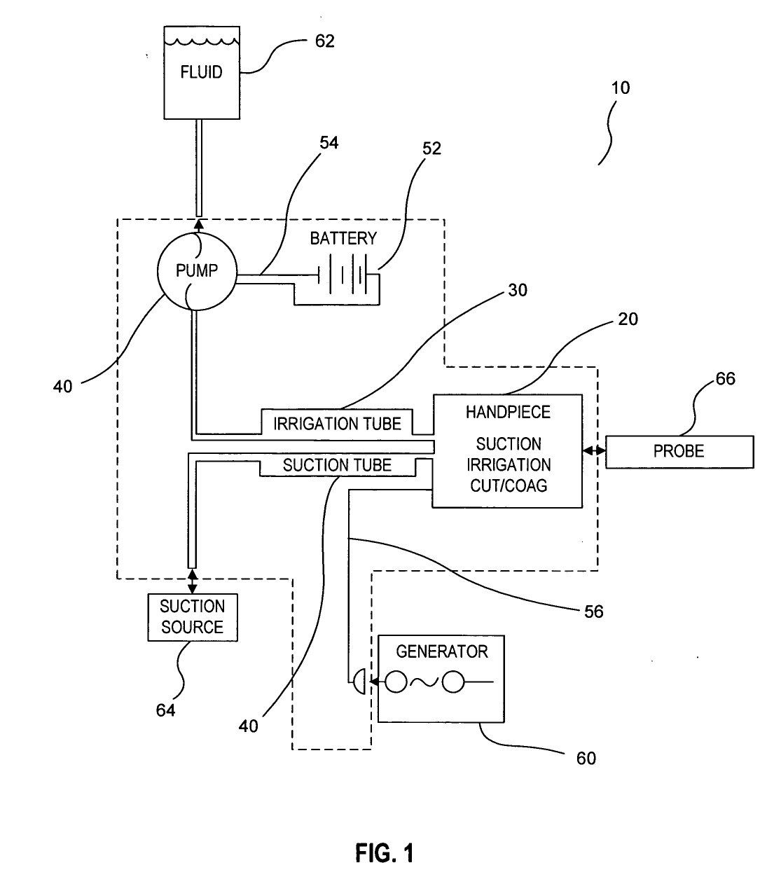

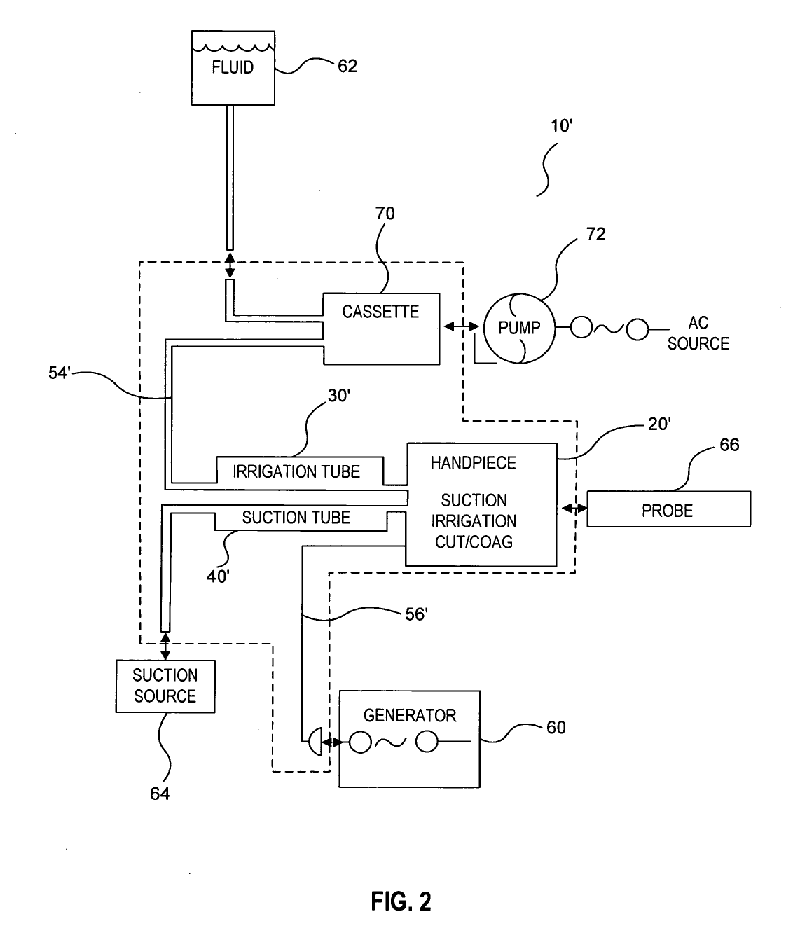

Two embodiments of an integrated, disposable suction-irrigation assembly of the invention are individually and schematically illustrated in FIGS. 1 and 2. In these figures, the dotted line forms a border surrounding the integrated, pre-assembled components of each embodiment, showing separation from the non-integrated components to which they can be coupled.

In one embodiment shown in FIG. 1, the invention provides an integrated, disposable suction-irrigation assembly 10 comprising a handpiece 20 having suction-irrigation-electrosurgical control functions and structure, irrigation tubing 30, suction tubing 40, a disposable pump 50, and a separate pump battery 52 in fixed, pre-assembled condition. The assembly also includes electrical conductors 54 (e.g., electrical wiring) for providing power to the pump 50 and electrical conductors 56 (e.g., electrical wiring) for providing electrosurgical energy to and through the handpiece 20. As seen in FIG. 1, the conductors 56 extend from the handpiece 20 and terminate in an adapter configured for connection to an electrosurgical generator 60, disposed outside the assembly 10.

Outside the dotted line designating the integrated, pre-assembled components of the assembly 10, FIG. 1 depicts an electrosurgical generator 60 configured for connection to (and providing energy through) conductors 56, a source of irrigation fluid 62, a suction source 64 (e.g., a vacuum source), and a medical treatment instrument 66 configured for connection with handpiece 20.

FIG. 2 depicts another arrangement for an integrated, disposable suction-irrigation assembly. FIG. 2 shows an integrated, disposable suction-irrigation assembly 10′ comprising a handpiece 20′ having suction-irrigation-electrosurgical control functions and structure, irrigation tubing 30′, suction tubing 40′, and a disposable pump-compatible cassette 70 in fixed, pre-assembled condition. In the embodiment of FIG. 2, the cassette 70 is disposable and configured for removable connection with a reusable AC-powered pump system 72. Therefore, in the embodiment of FIG. 2, the cassette 70 forms part of the assembly 10′ while the pump system 72 does not. For purposes of this disclosure, suction, irrigation, and electrosurgical control structure comprises structure of the handpiece that affects the suction, irrigation, and/or electrosurgical capabilities of the assembly.

As will be described in more detail below, the assembly 10′ also includes electrical conductors 54′ (e.g., electrical wiring) for connecting power from an AC-source, through the pump system 72, along the structure of cassette 70 and to the handpiece 20′ for actuation of the pump system 72 by an operator. The assembly 10′ further includes electrical conductors 56′ (e.g., electrical wiring) for providing electrosurgical energy to and through the handpiece 20′. Just as in FIG. 1, the conductors 56′ extend from the handpiece 20′ and terminate in an adapter configured for connection to an electrosurgical generator 60, disposed outside the assembly 10′.

Similar to the representation in FIG. 1, outside the dotted line designating the integrated, pre-assembled components of the assembly 10′, FIG. 2 depicts an electrosurgical generator 60 configured for connection to (and providing energy through) conductors 56′, a source of irrigation fluid 62, a suction source 64 (e.g., a vacuum source), and a medical treatment instrument 66 configured for connection with handpiece 20′.

The electrosurgical control functions for the handpieces 20 and 20′ can include cutting and coagulation functions. It has been discovered that suction-irrigation assemblies can be constructed such that a multitude of the required components can be manufactured for presentation in a fixed sub-assembly, and configured in a disposable arrangement. By providing cost effective disposable type arrangements, the need for costly decontamination and re-sterilization is obviated. As used herein, the term “integrated” is meant to refer to, for example, a pre-assembled state wherein the particular combination of components are presented to the user in a fixed, connected, or coupled condition established in advance during the manufacturing phase.

An integrated, disposable suction-irrigation assembly according to an embodiment of the invention is structured for connection to further components and equipment that would typically be required for its usage. For example, as illustrated in FIGS. 1-2, such components include electrosurgical generator 60, a source of irrigation fluid 62, a suction source 64, and a medical treatment instrument 66.

It has been discovered that the assembly disclosed herein is particularly useful within the context of monopolar electrosurgical systems that may accompany suction-irrigation systems. According to embodiments of the present disclosure, a single handpiece may include the combination of the electrosurgical system controls and fluid system controls. Such a handpiece can be referred to as a “multifunctional” handpiece. The term “multifunctional” when used to describe the capabilities of the handpiece is intended to include, for example, electrosurgical cutting and/or coagulation capabilities as well as suction and/or irrigation capabilities.

Moreover, the integrated, preassembled disposable assembly according to embodiments of the invention, is a “sub-assembly” relative to certain additional equipment to which it connects. The integrated, disposable suction-irrigation assembly according to embodiments of the invention facilitates attachment and accommodation of such additional equipment, ensures mechanical and electrical compatibility amongst the components included in the assembly, reduces set-up time for the procedure, and simplifies identification of the reusable components from the disposable components of the overall assembly used in the procedure.

Accordingly, an integrated, disposable suction-irrigation assembly according to embodiments of the invention in use is connected to additional equipment, including a fluid source or supply, a suction source, and generator or electrical power source (for the electrosurgical features). The connections of the assembly according to embodiments of the invention for such additional equipment can be universal or standard, thereby avoiding connective compatibility issues between the assembly and such additional equipment. For example, the tubing connectors and electrical connection structure (e.g., plugs) can be standard.

An integrated, disposable suction-irrigation assembly according to embodiments of the invention includes a suction-irrigation handpiece. Suction-irrigation handpieces that can be used include those that afford suction and irrigation control mechanisms. In a preferred embodiment of the invention, and an embodiment wherein the advantages of the invention can be fully realized and experienced, the suction-irrigation handpiece includes electrosurgical cut and coagulation control mechanisms in addition to suction and irrigation control mechanisms. Therefore, the entire assembly may be packaged for shipping in a package including all the integrated components in a single package. Accordingly, after a single use, the contents of the single package can then be disposed of. As one packaging example, all the components of the assembly may by placed within a basket having an open top surface. The top surface is then sealed with a covering enclosing the components of the assembly therein. The covering may enclose the contents with an airtight seal. The components of the assembly may be sterilized prior to placement within the basket.

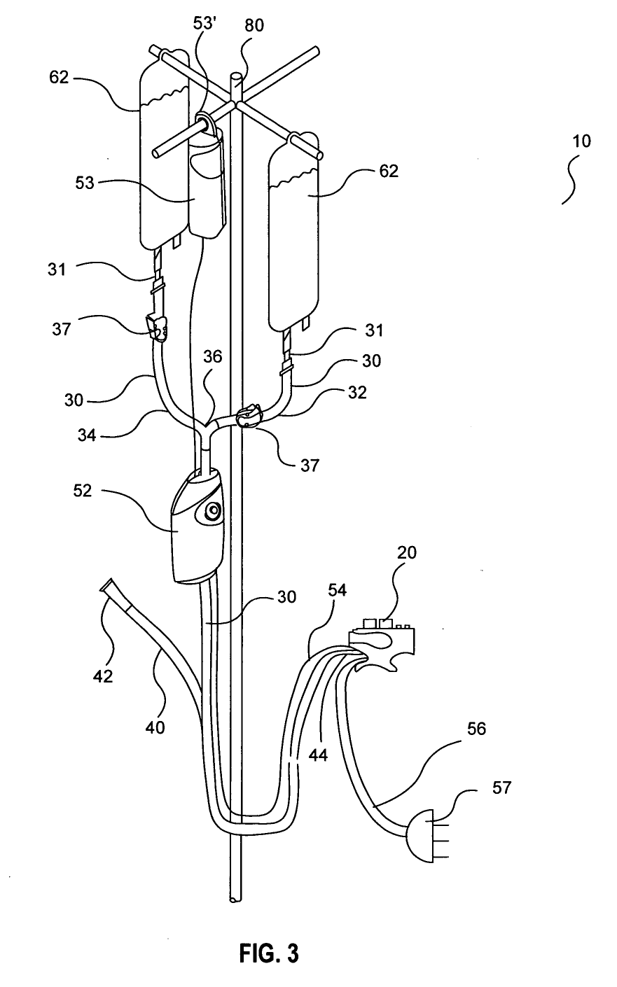

FIG. 3 depicts one example of a disposable assembly representing an embodiment corresponding to the schematic of FIG. 1. More particularly, FIG. 3 depicts an example of the components of an integrated, disposable suction-irrigation assembly 10. For example, FIG. 3, shows a multifunctional handpiece 20 having suction-irrigation-electrosurgical control functions. An IV pole 80 is provided to support some of the components of assembly 10 during use. For instance, irrigation tubing 30 is provided and is configured for connection at one end to a fluid source 62 (illustrated in this embodiment as I.V. bags housing saline). In the illustrated embodiment, two fluid sources 62 are depicted and each connects to a separate path 32 and 34, leading to a common tubing junction 36. The assembly may be provided with pinch clamps 37 for compressing the tubing and preventing fluid flow when desired. In addition, as seen in FIG. 3, for example, the irrigation tubing 30 may connect to fluid source(s) 62 by means of standard spike connectors 31 at the end of tubing 30.

Fluid sources that can be used in conjunction with the integrated, disposable suction-irrigation assembly 10 of the invention include containers, bags or other reservoir structures that are capable of tubing attachment or coupling thereto. Such fluid sources, include, but are not limited to, intravenous solution bags. Other fluid sources and containment or supply devices that are readily available to those in the medical field can also be used.

As seen in FIG. 3, the irrigation tubing 30 extends from the fluid sources 62 and connects at an input of a housing 52 for disposable pump 50. Additional irrigation tubing 30 exits an output from housing 52 for disposable pump 50 and continues to an input into the handpiece 20. As will be described in more detail below, in at least one embodiment, the handpiece 20 includes an irrigation control mechanism, whereby actuation of an irrigation button commences actuation of the pump and provides irrigation.

Suction tubing 40 is also depicted in FIG. 3. Suction tubing 40 extends from a first end 42, configured for connection to a source of suction 64, and leads to a second end 44, configured for connection to the handpiece 20. As will be described in more detail below, in at least one embodiment, the handpiece 20 includes a suction control mechanism, whereby actuation of a suction button commences suction. Suction sources that can be used in conjunction with the integrated, disposable suction-irrigation assembly of the invention include those connectible to tubing and suitable for providing a vacuum at a level compatible for the given procedure with which the invention is used. Suitable medical suction and vacuum equipment is readily available to those in the medical field and may include equipment typically available in hospital and other surgical settings.

With continued reference to FIG. 3, there is also described a pump battery pack 53 disposed a location separate from the disposable pump 50. The separate battery pack 53 can be provided with a hook 53′, thereby enabling the pack to suspend from the IV pole 80 during use. The battery pack 53 can provide power to the disposable pump 50 through electrical conductors 54 (e.g., illustrated as standard electrical wiring). The separation of the battery pack 53 from pump 50 provides at least two advantages. First, the weight of the pump 50 is substantially reduced by the illustrated arrangement since the battery pack is supported by virtue of its own suspension from the IV pole. Accordingly, the weight of the disposable pump 50 is substantially reduced, thereby reducing the pull on the irrigation fluid sources 62. Second, by separating the location of the disposable pump 50 and the battery pack 53, an operator can salvage the battery components from the assembly 10 instead of discarding them into a biohazard receptacle along with the pump 50 after use. In other words, the user can recover the batteries and put them to some alternative useful purpose, rather than simply disposing of them after a medical procedure (where useful battery power will most likely remain). Typically, the arrangement of batteries may comprise an array of 8 AA size units, though any suitable number and type to supply sufficient power may be used.

In use, the integrated, disposable suction-irrigation assembly 10 of FIG. 3, for example, is presented to the user for preparation and setting-up of the equipment for the medical procedure, such as laparoscopic surgery. Generally, where the assembly includes the integrated and disposable battery-powered pump 50, the irrigation tubing 30 can be attached to the fluid sources 62 through an irrigation spike connector, for example. The battery pack 52 can then be suspended from the IV pole 80 shared with the fluid sources 62. The electrical connection between battery pack 53 and the pump 50 can be confirmed by priming the pump 50 for fluid transport. Regarding the additional equipment to be used with the assembly 10, a treatment instrument 66 can then be connected to the handpiece 20, an electrosurgical connection plug (item 57 shown in FIG. 3, for example) can be connected to an electrosurgical generator (see FIGS. 1-2), and the suction tubing 40 can be connected to a suction source.

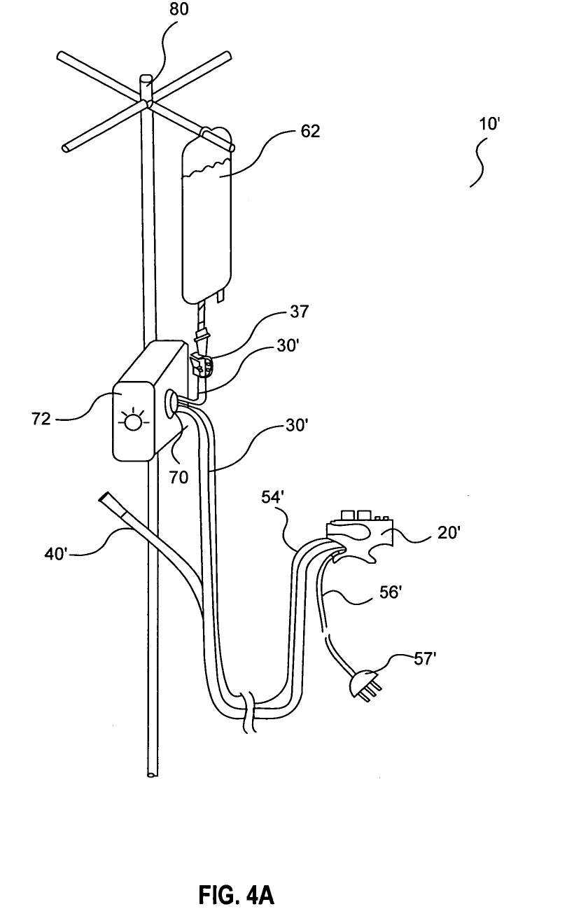

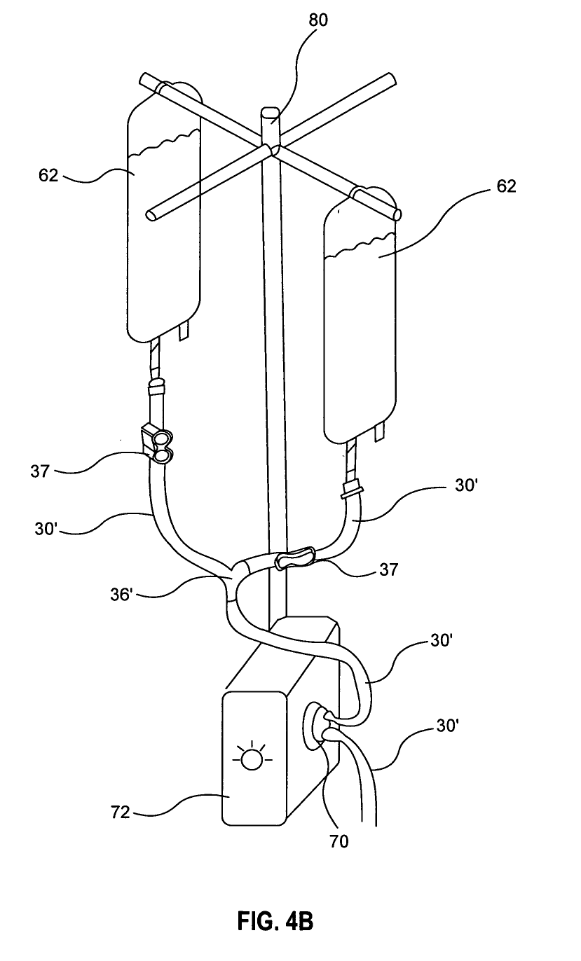

FIG. 4A, shows an example of a disposable assembly 10′ representing an embodiment corresponding to the schematic of FIG. 2. FIG. 4A depicts an example of the components of an integrated, disposable suction-irrigation assembly 10′. The example of an assembly 10′ in FIG. 4A differs from the arrangement depicted in FIG. 3 at least by the inclusion of a disposable pump-compatible cassette 70 and a reusable pump system 72, as opposed to the disposable pump 50. In the embodiment of FIGS. 2 and 4, the cassette 70 is disposable and configured for removable connection with a reusable AC-powered pump system 72. Accordingly, in the embodiment of FIG. 4A, the cassette 70 forms part of the disposable assembly 10′, while the pump system 72 does not. FIG. 4A illustrates the assembly 10′ using only one fluid source 62 connected to irrigation tubing 30′.

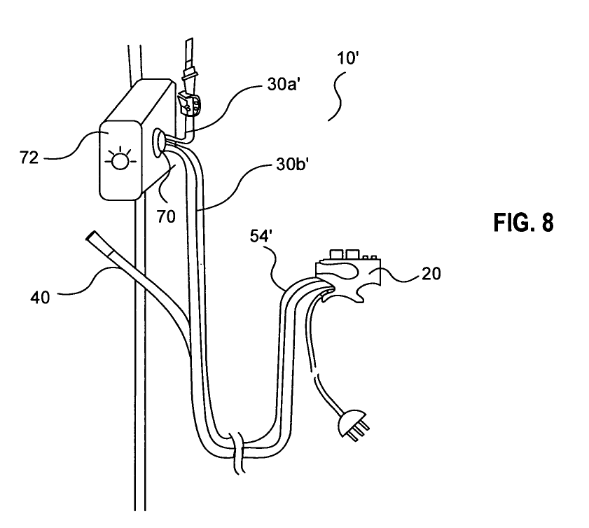

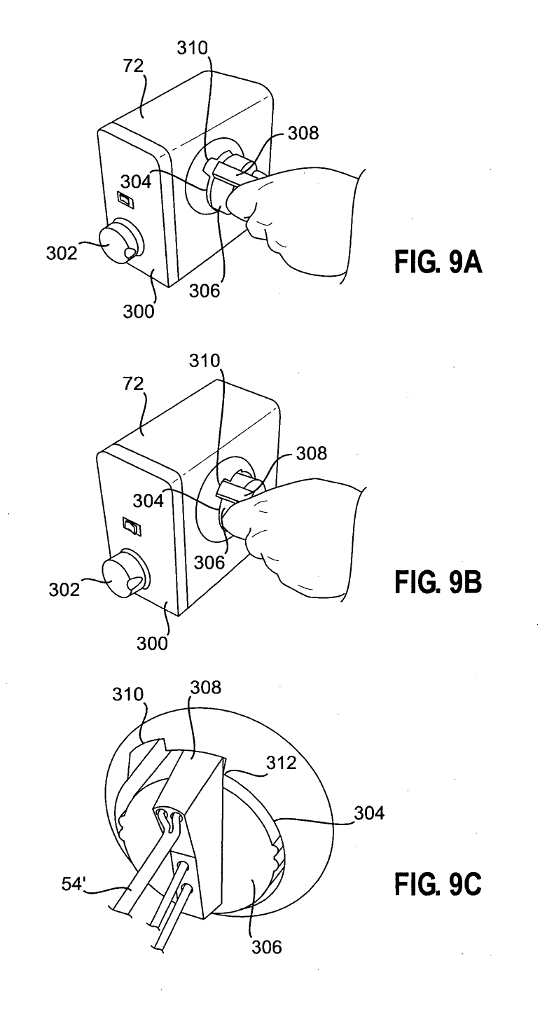

Other configurations are contemplated, however. For example, FIG. 4B depicts an assembly including at least two fluid sources 62 connected with a dual pathway irrigation tubing 30′ that leads to a common tubing junction 36′ located upstream of the pump 72. As will be described in more detail below with regard to FIGS. 8-9C, electrical power is provided to the pump system 72 via a standard AC wall plug. Power is then transferred from the pump system 72 via a connection made through the cassette 70 and along the irrigation tubing 30′ leading out of the pump and terminating at an actuation switch mechanism provided in the handpiece 20.

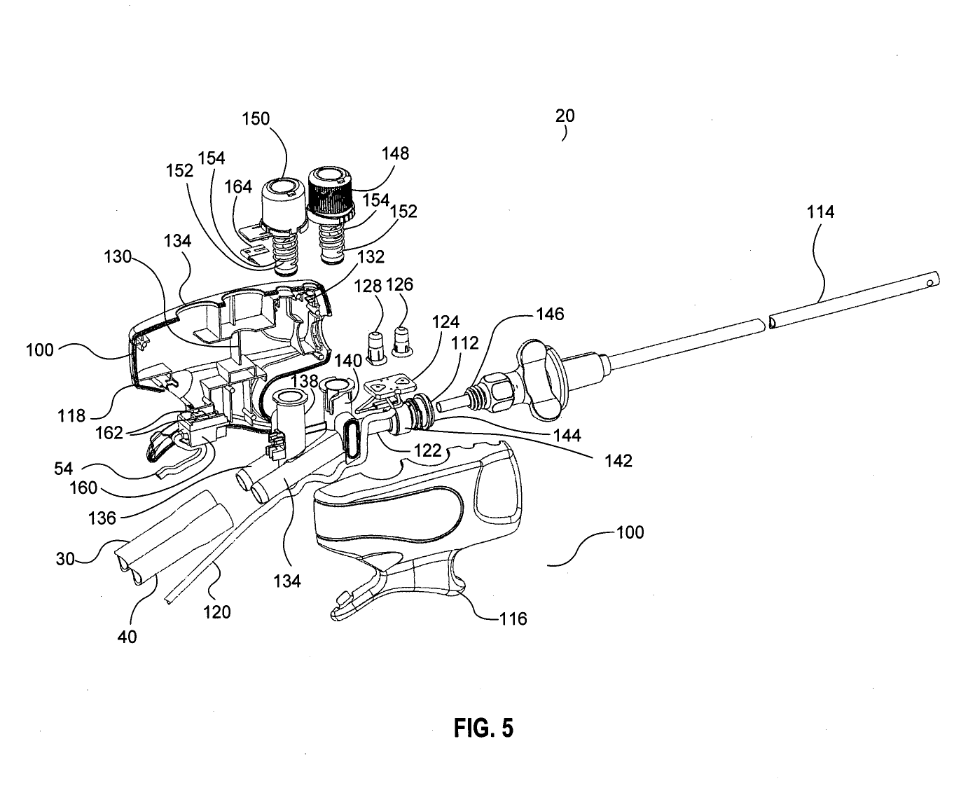

FIG. 5 discloses a disassembled perspective view of an exemplary medical device handpiece 20, according to an embodiment of the present disclosure. FIG. 5 illustrates one example of a multi-functional handpiece 20 having suction-irrigation and electrosurgical energy providing capabilities. The handpiece 20 is defined by a housing 100. One example for a particular shape, ergonomic features, and grasping enhancements exhibited by handpiece 20 is expressly described in co-pending U.S. application Ser. No. 11/526,872, filed on Sep. 26, 2006, the entire contents of which are hereby incorporated by reference.

As seen in FIG. 5, the housing 100 extends to an instrument attachment structure 112 within housing 100 for removably coupling a medical instrument 114 thereon. Incorporated by reference application Ser. No. 11/526,872 also includes a description of structure 112, its coupling to instrument 114, and various features of instrument 114, including its proximal collar. Reference is made to that application for that disclosure.

FIG. 5 depicts an exploded view of the internal and external structure of an exemplary handpiece 20. In FIG. 5, a first (right) housing half 116 is separated from a second (left) housing half 118. The first and second halves 116 and 118 each may be formed through a molding process such that they are configured to receive the internal components therebetween. During manufacture and assembly, the first and second halves 116, 118 may enclose the internal device components upon a mating engagement therebetween. For example, the interior of the first half 116 may include male pin mating protrusions configured for receipt within female pin apertures formed on the interior of the second half 118.

As seen in FIG. 5, the internal components of the handpiece 20 may include the internal electric wiring 120, a manifold assembly 122, and a conducting platform 124 for providing a transmission path for an electric circuit from wiring 120 to electrosurgical control buttons 126 and 128 (e.g., for controlling cutting and coagulation). Each half 116, 118 may include internal structure, such as preformed protrusions 130 defining separate chambers within the inside of each housing half, for receiving a particular component of the manifold assembly 122 and/or the wiring structure 120. For example, the second half 118 may include protrusions arranged to form a slot 132 configured to snugly receive the conducting platform 124 therein. In this configuration, the buttons 126, 128 are positioned to engage the platform 124 in order to complete an electric circuit thereby providing electric current via wiring 120 to the instrument 114. In addition, the handpiece 20 may include a semicircle configuration 134 for snugly receiving an exterior portion of the manifold assembly 122.

The wiring 120 that leads from the handpiece 20 is configured for connection with a source of electrosurgical energy, such as, an electrosurgical generator. Exemplary generators that can be used in conjunction with the integrated, disposable suction-irrigation assembly of the present disclosure include electrical generators structured for electrical connection to wiring 120 associated with an electrosurgical handpiece 20 through a pronged connector plug, for example. Examples of suitable electrical generators include LINVATEC™ Model 9700 and THE SYSTEM 5000™ (available from ConMed Corporation, Utica, N.Y.).

The manifold assembly 122 may include structure for routing suction and irrigation flow-paths in a predetermined configuration to a distal end of the handpiece 20 where the pathways connect to an appropriate conduit within a distal medical instrument 114. More particularly, the manifold assembly may comprise a proximal suction port 134 configured for connection to a portion of suction tubing 40 connected to a suction source 64 (or other suction generation), a proximal irrigation port 136 configured for connection to a portion of irrigation tubing 30 connected to an fluid irrigation source 62, an irrigation piston valve housing chamber 138, a suction piston valve housing chamber 140, and a distal manifold fluid conduit 142. The manifold assembly 122 is arranged such that proximal suction port 134 and proximal irrigation port 136 are capable of fluid communication with the distal manifold fluid conduit 142. The terminal portion of the distal manifold fluid conduit 142 may comprise an engagement portion 144 having internal threads configured to removably engage a medical instrument, such as instrument 114 via engagement with external threads 146, for example.

As to the medical instruments 114 to be coupled onto the handpiece 20, various instruments or probes can be provided with the handpiece. A variety of instruments and probes can further accompany the integrated, disposable suction-irrigation assembly of the invention, provided they are mechanically and/or electrically compatible with the handpiece 20 component of the assembly. The instruments or probes can be interchangeable and reusable or disposable. Thus, the instrument or probe attachment(s) for the handpiece 20 need not be pre-attached or integrated into the assembly according to embodiments of the invention. Examples of probes and instruments that can be used include those structured for delivery of irrigation-suction function to the surgical site, electrosurgical cutting and coagulation functions, and probes that can perform combinations of these and other functions, such as optic visualization. Non-limiting examples of instruments contemplated include a J-shaped probe, an L-shaped probe, a needle, a sphere shaped probe, and a surgical spatula.

As seen in FIG. 5, the medical device handpiece 20 includes a suction control button 148 and an irrigation control button 150. Each button 148 and 150 is engaged with a valve piston 152 and a compression spring 154. The working components of the handpiece 20 and the irrigation and suction control buttons 148 and 150 are expressly described in co-pending U.S. application Ser. No. 11/526,871, filed on Sep. 26, 2006, the entire contents of which are hereby incorporated by reference. As described in co-pending application Ser. No. 11/526,871, actuation of the control buttons 148 and/or 150 results in controlled actuation of suction and/or irrigation.

FIG. 5 also depicts a medical device including irrigation source power junction 160. As illustrated in FIG. 5, power junction 160 may present spaced apart electrically conductive contacts 162 on a top surface thereof. The power source junction 160 may be electrically connected to the disposable pump 50 of FIG. 3 or electrically connected to extend to electrical contacts on the cassette 70 for the pump 72 in FIG. 4A. As seen in FIG. 5, electrical connection can be made through electrical conductors 54 (e.g., electrical wiring) extending from power source junction 160 and extending to a power source for either pump 50 or pump 70.

The electrical conductors 54 can extend along the path of the tubing 30 and 30′. In one embodiment, the electrical conductors may be attached along the tubing 30 or 30′ with adhesive. Upon depressing irrigation control button 150, the conductive contact tangs 164 can contact the exposed spaced apart contacts 162 on the top surface of junction 160, thereby closing an open circuit and providing power to pump 50 or 70, for example.

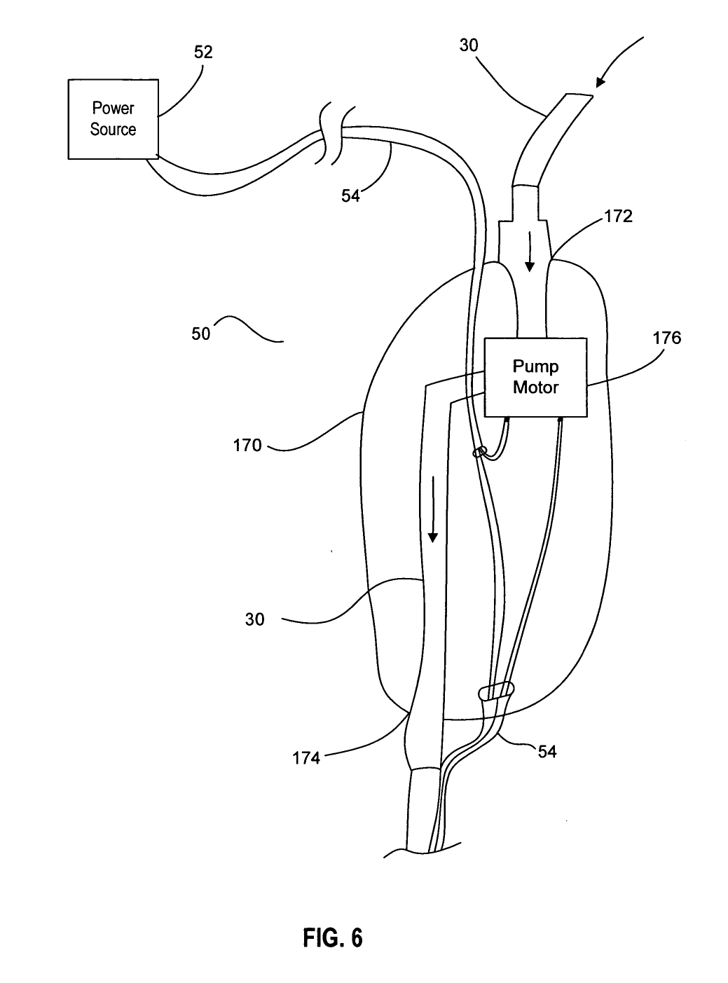

FIG. 6, discloses a schematic diagram depicting an exemplary battery powered pump 50 for use in an irrigation-suction assembly 10, according to an embodiment of the present disclosure. As seen in FIG. 6, the disposable pump 50 includes a housing 170 defining a fluid input opening 172 and a fluid output opening 174. The fluid input opening 172 provides a path for leading irrigation tubing 30 to an input of a pump/motor 176. During use, the pump/motor 176 receives fluid from the irrigation tubing 30 and drives fluid at an increased velocity out along the irrigation tubing that then extends out of the housing 170 through the fluid opening 174. As seen in FIG. 6, electrical power is provided to the pump/motor 176 from a remote battery pack 52 by way of electrical wiring 54. Additional wiring 54 exits the housing 170 near the fluid opening 174 and extends along the irrigation tubing until it enters the handpiece 20 and terminates at the power source junction 160.

As an alternative to the closed-circuit arrangement provided with junction 160, the activation (i.e., starting and stopping) of the pump 50 can be controlled by a variety of electrical, mechanical, or electro-mechanical switch mechanisms. For example, a float switch mechanism can be employed. With a float switch mechanism, fluid contacts a float switch inside the pump unit and moves the float switch a predetermined distance such that a photosensor detects one of two differing colors of the float. Based on the detected color (based on the position of the float switch), a motor containing an impellor is activated or deactivated. When activated, the motor drives the fluid through the irrigation tubing 30 and exits the pump 50 toward the handpiece 20. In this embodiment, the irrigation control of the handpiece 20 controls the movement of the float in the pump by virtue of contiguous fluid flow at the handpiece 50. Where a float switch is provided, the handpiece 20 does not include power junction 160 and electrical wiring 54 is not provided. Suitable pump systems that can be used include those similar to the HYDROSURGE PLUS™ system (available from C.R. Bard-Davol, Inc., Cranston, R.I.).

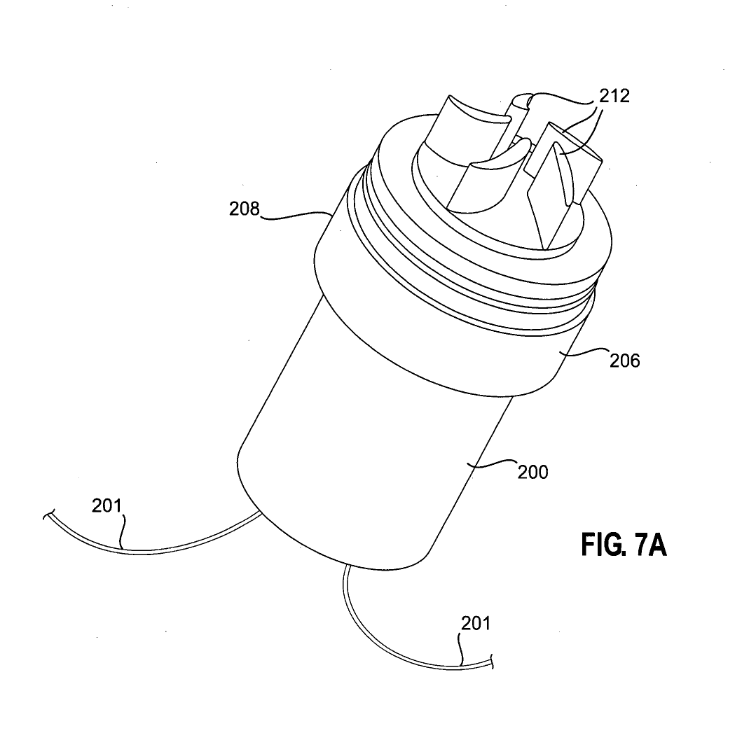

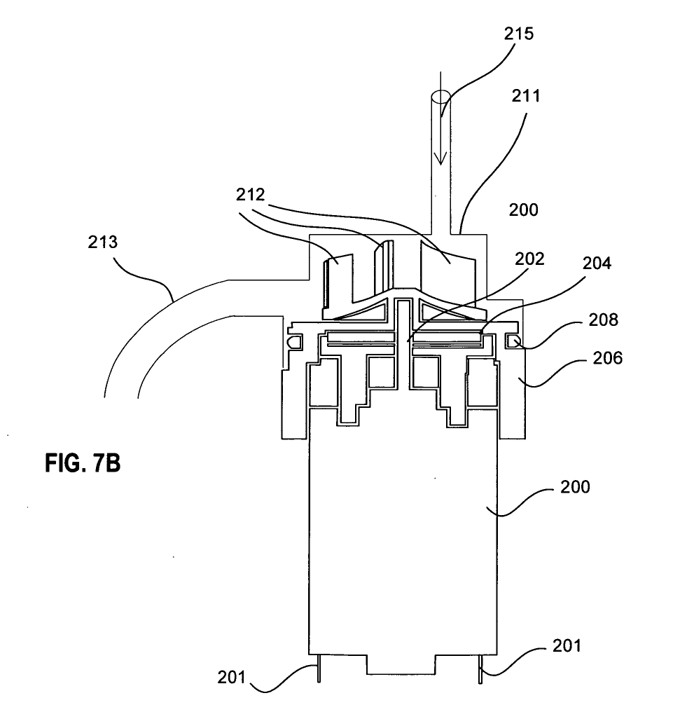

FIG. 7A depicts a perspective view of an exemplary motor/pump system for use in the battery powered pump of FIG. 6. In addition, FIG. 7B depicts a cross-sectional view of the exemplary motor/pump system illustrated in FIG. 7A. In the exemplary system of FIGS. 7A-7B, there is disclosed a motor body 200 having power leads 201. The motor body 200 drives a drive shaft 202. The drive shaft 202 extends from the motor body 200 through a gasket 204 and a top motor housing 206. An o-ring 208 extends along an exterior of the motor housing 206. The drive shaft 202 connects to, and drives via rotation, a fluid impeller 210 having concave shaped-ribs 212. During use, irrigation tubing 30 leads (as depicted by arrow 215) to a sealed fluid chamber that encloses the impeller 216 with a fluid chamber cap 211 sealed via the o-ring 208. As illustrated, the fluid input into the fluid chamber cap 211 can be offset from the central axis of the motor body 200 and the drive shaft 202. As seen in FIG. 7B, for example, the input portion of the fluid chamber cap is not located coaxially with the motor body 200 and the drive shaft 202. Upon priming the irrigation fluid and actuating motor body 200, the impeller 210 drives fluid to an exit 213 in the chamber cap 211 leading to further irrigation tubing 30 and the exit of the pump housing 170 of FIG. 6. The disclosed motor/pump system of FIGS. 7A-7B is intended merely as an example and other pump configurations are contemplated.

FIG. 8 depicts an AC-powered pump and a cassette compatible therewith for an alternative exemplary pumping system according to an embodiment of the present disclosure. In addition, FIGS. 9A-9C depict connection of the cassette 70 of FIG. 8 providing an irrigation flowpath to an AC-powered pump. FIG. 8 discloses a disposable pump-compatible cassette 70 and a reusable pump system 72, as opposed to the disposable pump 50. In the embodiment of FIG. 8, the cassette 70 is disposable and configured for removable connection with a reusable AC-powered pump system 72. In general, the cassette 70 can comprise a casing that includes input and output attachments for irrigation tubing 30, and a flexible reciprocating diaphragm in communication with the fluid passageway within the casing. The reciprocating diaphragm can affect the forcing of fluid through the input irrigation line which is coupled to a fluid source 62, and exiting the output irrigation line toward the irrigation control mechanism of the handpiece 20. One-example of a suitable cassette-pump system that can be used in conjunction with embodiments of the invention is the PULSEWAVE™ system (available from Cardinal Health, Inc., Dublin, Ohio).

As seen in FIGS. 9A-9C, pump system 72 is configured for connection to an AC-power supply and includes a pump housing 300 having a pump speed control dial 302. In addition, the pump housing 300 defines an aperture 304 for receiving the cassette 70. Cassette 70 is provided at an intermediate location along the irrigation tubing 30′. As seen in FIG. 8, a sub-portion of the irrigation tubing 30′, portion 30a′, extends between the fluid source 62 and the cassette 70. In addition, another sub-portion of irrigation tubing 30′, portion 30b′, extends between the cassette 70 and the handpiece 20.

As seen in FIG. 9A, cassette 70 includes a terminal collar 306 configured for connection with the pump housing 300. The collar 306 includes an exterior shape compatible with the shape of the aperture 304. Accordingly, a fluid flowpath from tubing portion 30a′ into housing 300 and out of housing 300 entering into tubing portion 30b′ is established when collar 306 is engaged with aperture 304. For example, the collar 306 may include a rectangular protrusion 308 configured for alignment with a corresponding recess 310 defined by the aperture 304.

In order to enable fluid irrigation capabilities for assembly 10′, a user need only appropriately align the collar 306 with the aperture 304 as seen in FIG. 9A, insert the collar 306 into the aperture 304 as seen in FIG. 9B, and then partially rotate the collar 306 in a clockwise direction relative to the aperture 304, as seen in FIG. 9C. Final rotation is completed (and further rotation is prevented) when the protrusion 308 abuts against a stop 312 defined by the aperture 304. When final rotation is completed, an irrigation fluid flow path is provided, leading from a fluid source 62 and extending to the handpiece 20.

As seen in FIG. 8 and 9C, cassette 70 and tubing portion 30b′ includes conductors 54 in the form of electrical wiring for connection with power junction 160 in the handpiece 20, for example. Accordingly, AC power within the housing 300 is provided to the conductors 54′ upon engagement of collar 306 and aperture 304. Therefore, in addition to providing a fluid path, engagement of collar 306 and aperture 304 also provides electrical connection of AC power to the conductors 54′. In the illustrated embodiment, the electrical connection can run alongside tubing portion 30b′ directly to the cassette 70, which in turn includes the electrical connection to the pump unit 72. Thus, the irrigation control of the handpiece 20 can electrically activate and initiate pump operation thereby controlling the irrigation fluid flow. Non-limiting examples for the establishment of an electrical connection include an electrical ball and socket arrangement, a pin and socket arrangement, and an extending prong and corresponding contact platform arrangement.

In all of the embodiments described above, an important aspect of the disclosure is that the tubing 30 and 40 and much of the associated equipment is pre-attached and eliminates the need for the user to modify the equipment for multifunctional suction-irrigation-electrosurgical systems. Furthermore, inter-component compatibility is assured at least within the integrated, disposable assembly of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An integrated, disposable medical device assembly, comprising:
a handpiece having suction, irrigation, and electrosurgical control structure;
irrigation tubing configured for connection to an irrigation fluid source at one end and connected to the handpiece at another end;
suction tubing configured for connection to a source of suction at one end and connected to the handpiece at another end;
a disposable fluid pump located along a path of the irrigation tubing;
a power pack electrically coupled to the disposable fluid pump to provide power to the disposable fluid pump; and
electrical wiring leading from the handpiece and configured for connection to a source of electrosurgical energy.

2. The assembly of claim 1, wherein the irrigation tubing is configured for connection to at least two fluid sources by way of a dual pathway irrigation tubing that leads to a common tubing junction located upstream of the disposable pump.

3. The assembly of claim 1, further comprising at least one pinch clamp configured for preventing fluid flow along the suction tubing or irrigation tubing.

4. The assembly of claim 1, wherein the power pack includes a hook configured for hanging the power pack on an IV pole during use.

5. The assembly of claim 1, wherein electrical wiring extends from the disposable pump and leads to conductive contacts of the handpiece.

6. The assembly of claim 5, wherein the handpiece includes an irrigation control button including at least one conductive contact such that actuation of the irrigation button closes an electric circuit, thereby providing power to the disposable pump.

7. The assembly of claim 5, wherein the electrical wiring extending from the disposable pump runs along an exterior surface of the irrigation tubing.

8. The assembly of claim 1, wherein the suction tubing is united alongside the irrigation tubing.

9. The assembly of claim 1, wherein electrical wiring transmits power from the power pack to the disposable fluid pump.

10. The assembly of claim 1, wherein the disposable fluid pump includes a housing that surrounds a motor body including a drive shaft that rotationally drives a fluid impellor.

11. The assembly of claim 10, wherein the impellor includes a plurality of concave shaped ribs.

12. The assembly of claim 1, wherein the handpiece includes an irrigation control button, and wherein a portion of the irrigation tubing entering the disposable pump includes a float switch and photosensor arrangement such that actuation of the irrigation control button primes a fluid supply and affects movement of the float switch, thereby activating the disposable pump.

13. The assembly of claim 1, wherein the power pack is provided at a location separate from disposable fluid pump.

14. The assembly of claim 1, wherein the power pack includes at least one disposable battery.

15. The assembly of claim 1, wherein the power pack includes a first housing and the disposable fluid pump includes a second housing distinct from the first housing.

16. An integrated, disposable medical device assembly, comprising:
a handpiece having suction, irrigation, and electrosurgical control structure;
irrigation tubing configured for connection to an irrigation fluid source at one end and connected to the handpiece at another end;
suction tubing configured for connection to a source of suction at one end and connected to the handpiece at another end;
a disposable fluid pump cassette located along a path of the irrigation tubing; and
electrical wiring leading from the handpiece and configured for connection a source of electrosurgical energy.

17. The assembly of claim 16, wherein the irrigation tubing is configured for connection to at least two fluid sources by way of a dual pathway irrigation tubing that leads to a common tubing junction located upstream of the cassette.

18. The assembly of claim 16, further comprising at least one pinch clamp configured for preventing fluid flow along the suction tubing or irrigation tubing.

19. The assembly of claim 16, wherein the cassette is configured for connection with a reusable pump system such that during use, fluid from the fluid source flows through the cassette, into the pump system, out of the pump system, through the cassette again, and to the handpiece via the irrigation tubing.

20. The assembly of claim 16, wherein the pump system is powered by an AC power source.

21. The assembly of claim 16, wherein the cassette includes electrical contacts configured to establish electrical connection to corresponding contacts in the reusable pump system.

22. The assembly of claim 21, wherein the electrical contacts of the cassette extend to electrical wiring that leads to the handpiece, and wherein the handpiece includes an irrigation control button including at least one conductive contact such that actuation of the irrigation button closes an electric circuit, thereby providing power to the pump system.

23. The assembly of claim 21, wherein the electrical wiring extending from the cassette runs along an exterior surface of the irrigation tubing between the cassette and the handpiece.

24. The assembly of claim 16, wherein the cassette includes a collar configured for engagement with an aperture of the reusable pump system upon rotation of the collar within the aperture.

25. A method of assembling and using a medical device comprising:
removing from packaging, an integrated, disposable medical device assembly, the medical device assembly comprising:
a handpiece having suction, irrigation, and electrosurgical control structure;
irrigation tubing configured for connection to an irrigation fluid source at one end and connected to the handpiece at another end;
suction tubing configured for connection to a source of suction at one end and connected to the handpiece at another end;
a disposable fluid pump located along a path of the irrigation tubing;
a power pack electrically coupled to the disposable fluid pump to provide power to the disposable fluid pump; and
electrical wiring leading from the handpiece and configured for connection a source of electrosurgical energy;
connecting the irrigation tubing to an irrigation fluid source;
connecting the suction tubing to a source of suction;
connecting the wiring to an electrosurgical generator;
using the assembly to provide irrigation, suction, and electrosurgical treatment in a medical procedure; and
disposing of the medical device assembly.

26. The method of claim 25, wherein using the assembly to provide suction includes actuating a suction button on the handpiece.

27. The method of claim 25, wherein using the assembly to provide irrigation includes actuating an irrigation button on the handpiece.

28. The method of claim 25, wherein using the assembly comprises actuating electrosurgical energy with the handpiece and then actuating suction with the handpiece.

29. The method of claim 25, wherein at least one battery of the power pack is removed from the medical device assembly prior to disposing of the medical device assembly.

30. The method of claim 25, wherein the medical device assembly is disposed of after a single use.

31. The method of claim 25, further comprising connecting an instrument to the handpiece, wherein the instrument comprises one of a J-shaped probe, an L-shaped probe, a needle probe, a sphere shaped probe, and a surgical spatula probe.

32. The method of claim 25, wherein connecting the irrigation tubing to an irrigation fluid source includes inserting a connector into a saline bag.

33. A method of assembling and using a medical device comprising:
removing from packaging, an integrated, disposable medical device assembly, the medical device assembly comprising:
a handpiece having suction, irrigation, and electrosurgical control structure;
irrigation tubing configured for connection to an irrigation fluid source at one end and connected to the handpiece at another end;
suction tubing configured for connection to a source of suction at one end and connected to the handpiece at another end;
a disposable fluid pump cassette located along a path of the irrigation tubing; and
electrical wiring leading from the handpiece and configured for connection a source of electrosurgical energy;
connecting the irrigation tubing to an irrigation fluid source;
connecting the suction tubing to a source of suction;
connecting the wiring to an electrosurgical generator;
connecting the cassette to a reusable pump system powered by an AC power source;
using the assembly to provide irrigation, suction, and electrosurgical treatment in a medical procedure; and
disposing of the medical device assembly.

34. The method of claim 33, wherein the pump is reused in another medical procedure using a new assembly.

35. The method of claim 33, wherein using the assembly to provide suction includes actuating a suction button on the handpiece.

36. The method of claim 33, wherein using the assembly to provide irrigation includes actuating an irrigation button on the handpiece.

37. The method of claim 33, wherein using the assembly comprises actuating electrosurgical energy with the handpiece and then actuating suction with the handpiece.

38. The method of claim 33, wherein the medical device assembly is disposed of after a single use.

39. The method of claim 33, further comprising connecting an instrument to the handpiece, wherein the instrument comprises one of a J-shaped probe, an L-shaped probe, a needle probe, a sphere shaped probe, and a surgical spatula probe.

40. The method of claim 33, wherein connecting the irrigation tubing to an irrigation fluid source includes inserting a connector into a saline bag.

41. A method of packaging and shipping a disposable medical device comprising:
packaging an integrated, disposable medical device assembly, the medical device assembly comprising:
a handpiece having suction, irrigation, and electrosurgical control structure;
irrigation tubing configured for connection to an irrigation fluid source at one end and connected to the handpiece at another end;
suction tubing configured for connection to a source of suction at one end and connected to the handpiece at another end;
a disposable fluid pump cassette located along a path of the irrigation tubing; and
electrical wiring leading from the handpiece and configured for connection a source of electrosurgical energy;
enclosing the assembly within packaging; and
shipping the enclosed assembly to a supplier or user.

42. The method of claim 41, wherein packaging the assembly includes placing the assembly within a basket having an open top surface and sealing the top surface of the covering.

43. The method of claim 42, wherein the basket is enclosed with an airtight seal.

44. The method of claim 41, wherein the assembly is sterilized prior to shipping the assembly.
